(19) United States
(12) Plant Patent Application Publication
(10) Pub. No.: US 2017/0112040 P1
(43) Pub. Date: Apr. 20, 2017

(54) LACHENALIA JACQ. F. EX MURRAY named "RAINBOW BELLS"

(50) ;

(71) Applicant: AGRICULTURAL RESEARCH COUNCIL, Pretoria (ZA)

(72) Inventor: Riana Kleynhans, Benoni (ZA)

(21) Appl. No.: 14/756,821

(22) Filed: Oct. 19, 2015

(51) Int. Cl. A01H 5/00

(52) U.S. Cl. PLT/263.1

(57) ABSTRACT

A new and distinct form of Lachenalia plant characterized by a bulb plant type having a spreading growth habit of a height of 15-30 centimetres (cm) and width of 10-15 cm; a bell-shaped multicolored yellow, orange and red flower.

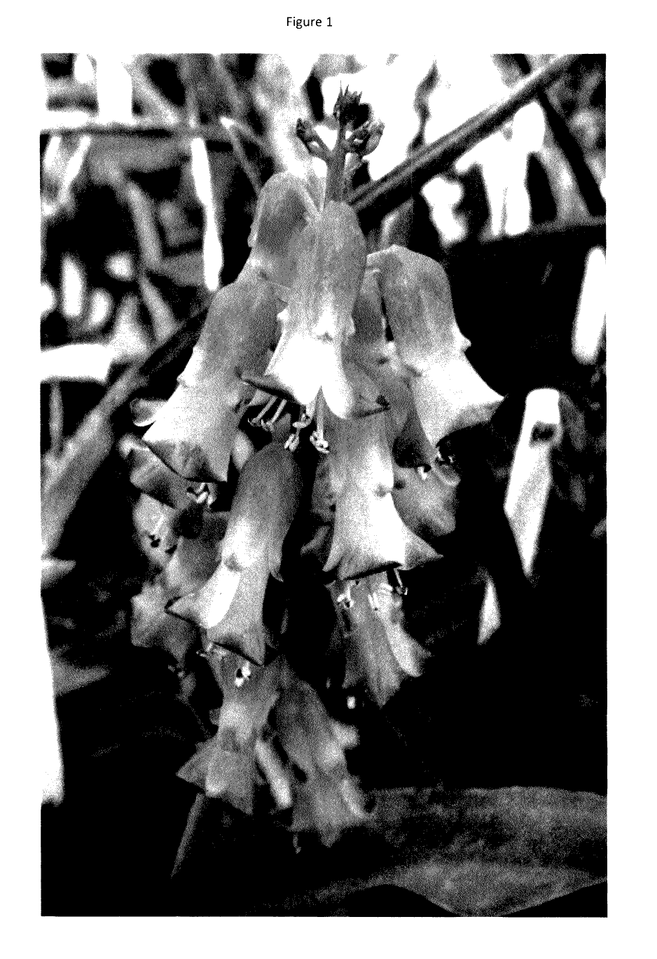

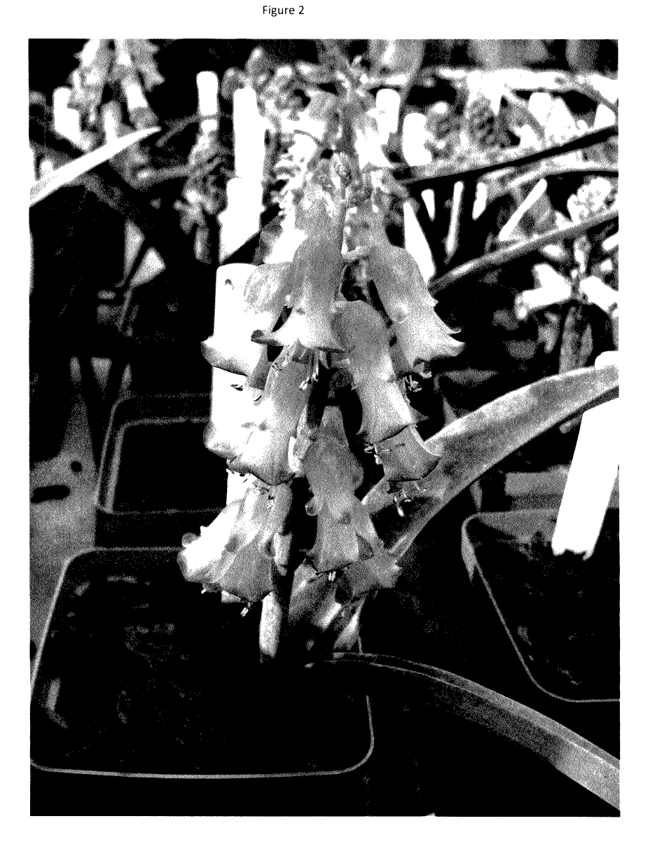

Genus and species: Lachenalia Jacq. f. ex Murray.

Variety denomination: Rainbow Bells.

### BACKGROUND OF THE INVENTION

The present invention relates to a new and distinct cultivar of Lachenalia, botanically known as Lachenalia Jacq. f. ex Murray having the common name of ViooltjieslCape cowslip and hereinafter referred to by its variety denomination “Rainbow Bells”.

The variety was first cultivated in 2008 and originated by means of conventional breeding by the inventor during 2009 to 2010 at the premises of Agricultural Research Council Vegetable and Ornamental Plant Institute, Roodeplaat, Pretoria, Republic of South Africa.

Asexual reproduction of the Rainbow Bells plant has shown that the unique features thereof are stable and reproduced true to type in successive generations.

### SUMMARY OF THE INVENTION

The present invention constitutes a new and distinct variety of Lachenalia which originated from an inter-species cross between two different accessions of Lachenalia flava.

The following traits have been repeatedly observed and are determined to be the unique characteristics, under the local evaluation conditions, of the Rainbow Bells plant. These characteristics in combination distinguish Rainbow Bells as a new and distinct Lachenalia cultivar:
1. Bulb plant type having a spreading growth habit of a height of 15-30 centimetres (cm) and width of 10-15 cm.
2. Flower color: red when opening, yellow when open; multicolored yellow, orange and red.
3. Flower form: Bell shaped.

Plants of the new Lachenalia variety Rainbow Bells can be compared to similar denominations such as Romelia and Namakwa. Romelia has plain yellow bell shaped flowers whereas Rainbow Bells has multicolored yellow, orange and red bell shaped flowers. Namakwa has plain yellow with red tip flowers of tubular shape.

### BRIEF DESCRIPTION OF THE PHOTOGRAPHS

The accompanying colored photographs illustrate the overall appearance of the Rainbow Bells, showing the colors as true as it is reasonably possible to obtain in colored reproductions of this type. The photographs comprises front perspective views of a typical Rainbow Bells grown in an indoor nursery.

### DETAILED DESCRIPTION

The aforementioned photographs, following observations and measurements describe Rainbow Bells grown by the inventor on behalf of Agricultural Research Council Vegetable and Ornamental Plant Institute, Roodeplaat, Pretoria, Republic of South Africa. Thereafter Rainbow Bells was made subject of a South African Plant Breeders' Right application in March 2009. This application was granted in 2010 under number ZA20104396.

The plant may be used for landscaping and should be planted during the month of March; indoors and in pots only. The plant should be kept at temperatures of 20-25 degrees Celsius during the day and 10-15 degrees Celsius at night. Plenty of light should be provided.

Rainbow Bells is an early flowering cultivar with an exceptional keeping quality.
Leaf:
   Attitude.—Semi-erect.
   Length.—Medium — 20 cm.
   Width.—Medium — 3.7 cm.
   Ratio length/width.—Large (elongated).
   Shape.—Oblong.
   Color.—Light green.
   Shape in cross section.—Angular.
   Recurving and undulation of margin.—Absent.
   Blistering of upper side.—Absent.
   Spots and pubescence of upper side.—Absent.
   Markings on outer side of leaf base.—Absent.
Peduncle:
   Length.—Medium — 13 cm.
   Spots or markings.—Absent.
Interflorescense:
   Length.—Short to medium — 8.5 cm.
   Rudimentary apex.—Inconspicuous.
   Number of flowers.—Few to medium.
Flower:
   Attitude.—Pendulous.
   Pedicel.—Present.
   Length of pedicel.—Medium.
   Flower length.—Medium — 2.6 cm.
   Diameter excluding apex.—Medium to large — 1.1 cm.
   Diameter at apex.—Medium to large — 1.6 cm.
   Attitude of distal pan of inner perianth segments.—Spreading.
   Glossiness.—Present.
   Predominant color.—Orange-yellow.
   Intensity of predominant color.—Strong.
   Outer perianth segment.—color of basal part just before opening of flower. Orange-red (RHS #34A).
   Outer perianth segment.—color of basal part of fully opened flower. Yellow-orange (RHS #17B).
   Outer perianth segment.—color of apex relative to other part. Clearly different.
   Outer perianth segment.—color of apex if clearly different from other part. Yellow-green.
   Inner perianth segment.—distinct spot in middle of apex. Absent.
   Inner perianth segment.—color of margin of apex relative to other part. Clearly different.
   Inner perianth segment.—color of differently colored margin of apex. Brown.
   Inner perianth segment.—predominant color of exposed part (excluding differently colored margin of apex). Yellow-orange (RHS #17C).
   Length of inner perianth segment compared to outer segments.—Much longer.
   Extrusion of stamens.—Present.
   Fragrance.—Absent.
   Bulb.—predominant shape Oblate.
   Time of flowering.—Very early.
   Duration of flowering.—Medium. The variety is not genetically modified and has been tested to be free of Ornithogalum Mosaic Virus.

The invention claimed is:

1. A new and distinct cultivar of Lachenalia named Rainbow Bells as illustrated and described herein.
